(19) United States
(12) Patent Application Publication
(10) Pub. No.: US 2022/0287366 A1
(43) Pub. Date: Sep. 15, 2022

(54) VAPORIZER DEVICE WITH IMPROVED HEATER

(71) Applicant: JUUL Labs, Inc., San Francisco, CA (US)

(72) Inventor: Alexander Weiss, Oakland, CA (US)

(21) Appl. No.: 17/638,678

(22) PCT Filed: Aug. 28, 2020
(86) PCT No.: PCT/US20/48462

§ 371 Date: Feb. 25, 2022

Related U.S. Application Data
(60) Provisional application No. 62/894,554, filed on Aug. 30, 2019.

(51) Int. Cl. A24F 40/46; H05B 7/20; A24F 40/485; A24F 40/57; A24F 40/65

(52) U.S. Cl. CPC A24F 40/46; H05B 7/20; A24F 40/485; A24F 40/57; A24F 40/65; H05B 2203/021; A61M 15/06

(57) ABSTRACT

Features relating to a vaporizer device including a reusable vaporizer device body and a cartridge with a reservoir containing a vaporizable material are provided. Aspects of the current subject matter provide for improved heating of a vaporizable material stored within the vaporizer device by reducing the amount of time to re-saturate the capillary pathway, improving the consistency of vaporizable material that is vaporized, enhancing the ability to control a precise amount of vaporizable material that is vaporized, and improving the ability to monitor the amount of vaporizable material that is vaporized.

### CROSS-REFERENCE TO RELATED APPLICATIONS

This application claims priority to U.S. Provisional Application No. 62/894,554, filed on Aug. 30, 2019, and titled “Vaporization Device with Improved Heater,” the entirety of which is incorporated by reference herein.

### TECHNICAL FIELD

The current subject matter described herein relates generally to vaporizer devices, such as portable, personal vaporizer devices for generating and delivering an inhalable aerosol from one or more vaporizable materials, and more particularly relates to heating in a vaporizer device.

### BACKGROUND

Vaporizing devices, including electronic vaporizers or e-vaporizer devices, allow the delivery of vapor and aerosol containing one or more active ingredients by inhalation of the vapor and aerosol. Electronic vaporizer devices are gaining increasing popularity both for prescriptive medical use, in delivering medicaments, and for consumption of nicotine, tobacco, other liquid-based substances, and other plant-based smokeable materials, such as cannabis, including solid (e.g., loose-leaf or flower) materials, solid/liquid (e.g., suspensions, liquid-coated) materials, wax extracts, and prefilled pods (cartridges, wrapped containers, etc.) of such materials. Electronic vaporizer devices in particular may be portable, self-contained, and convenient for use.

### SUMMARY

Aspects of the current disclosure relate to heating in a vaporizer device, such as in a cartridge of a vaporizer device, for improved aerosol production.

According to some aspects, a vaporizer device may include a reservoir configured to store a vaporizable material and a heater configured to vaporize the vaporizable material stored in the reservoir. The heater may include a nozzle, a heating element, and a capillary pathway. The nozzle may include a channel extending along a central longitudinal axis. The channel may direct the vaporized vaporizable material to an outlet of the vaporizer device. The heating element may include a pair of conductive elements that are separated by an opening. The opening may be axially aligned with the channel along the central longitudinal axis. The pair of conductive elements being oppositely charged. The capillary pathway may be defined by the channel and the opening. The capillary pathway may draw and hold the vaporizable material. An electric arc may form between the pair of conductive elements, thereby vaporizing at least some of the vaporizable material held within the capillary pathway.

In some aspects, the vaporizer device also includes a plurality of heaters positioned in a patterned array. In some aspects, the plurality of heaters includes a first heater and a second heater. Power may be supplied sequentially to the first heater and the second heater such that the second heater vaporizes the vaporizable material held within the capillary pathway of the second heater after the first heater vaporizes at least some of the vaporizable material held within the capillary pathway of the first heater. In some aspects, the opening defines a passageway formed through a thickness of the heat transfer substrate.

In some aspects, the heater further includes a nozzle substrate and a heat transfer substrate. A plurality of nozzles may be positioned on the nozzle substrate and a plurality of heating elements may be positioned on the heat transfer substrate.

In some aspects, the nozzle abuts the heating element.

According to some aspects, a heater for a vaporizer device configured to vaporize vaporizable material stored within a reservoir of the vaporizer device may include a nozzle, a heating element, and a capillary pathway. The nozzle may include a channel extending along a central longitudinal axis. The channel may direct vaporized vaporizable material to an outlet of the vaporizer device. The heating element may include a pair of conductive elements that are separated by an opening. The opening may be axially aligned with the channel along the central longitudinal axis. The pair of conductive elements may be oppositely charged. The capillary pathway may be defined by the channel and the opening. The capillary pathway may draw and hold the vaporizable material. An electric arc may be formed between the pair of conductive elements, thereby vaporizing at least some of the vaporizable material held within the capillary pathway.

According to some aspects, a vaporizer device includes a reservoir configured to store a vaporizable material and a heater configured to vaporize the vaporizable material stored in the reservoir. The heater may include a nozzle, a heating element, a printed circuit board coupled to and supporting the heating element, and a capillary pathway. The nozzle may include a channel extending along a central longitudinal axis. The channel may direct vaporized vaporizable material to an outlet of the vaporizer device. The heating element may be spaced apart from the nozzle by a gap. The heating element may include a resistive element configured to generate heat. The heating element may be axially aligned with the channel along the central longitudinal axis. The printed circuit board may be coupled to and support the heating element. The printed circuit board may include a controller configured to control the generation of heat by the heating element. The capillary pathway may be defined by the channel and the gap. The capillary pathway may draw and hold the vaporizable material. The heat generated by the heating element may be transferred to the vaporizable material held within the capillary pathway, thereby causing the vaporization of at least some of the vaporizable material.

According to some aspects, a method of vaporizing a vaporizable material stored within a vaporizer device may include supplying power to a first heater of the vaporizer device, thereby causing the vaporizable material in thermal contact with the first heater to be vaporized. The method may also include supplying power to a second heater of the vaporizer device after at least some of the vaporizable material in thermal contact with the first heater has been vaporized, thereby causing the vaporizable material in thermal contact with the second heater to be vaporized.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings:

FIG. 1A-FIG. 1F illustrate features of a vaporizer device including a vaporizer body and a cartridge consistent with implementations of the current subject matter;

FIG. 2 is a schematic block diagram illustrating features of a vaporizer device having a cartridge and a vaporizer body consistent with implementations of the current subject matter;

FIG. 3 schematically illustrates a side view of an example heater of a vaporizer device consistent with implementations of the current subject matter;

FIG. 4 schematically illustrates a top view of an example heater of a vaporizer device consistent with implementations of the current subject matter;

FIG. 5 schematically illustrates an exploded view of an example heater of a vaporizer device consistent with implementations of the current subject matter;

FIG. 6 schematically illustrates a side view of an example heater of a vaporizer device consistent with implementations of the current subject matter;

FIG. 7 schematically illustrates a top view of an example heater of a vaporizer device consistent with implementations of the current subject matter;

FIG. 8 schematically illustrates an exploded view of an example heater of a vaporizer device consistent with implementations of the current subject matter; and

FIG. 9 is an example method of heating a vaporizable material consistent with implementations of the current subject matter.

When practical, similar reference numbers denote similar structures, features, or elements.

### DETAILED DESCRIPTION

Implementations of the current subject matter include devices relating to vaporizing of one or more materials for inhalation by a user. The term “vaporizer” may be used generically in the following description and may refer to a vaporizer device, such as an electronic vaporizer. Vaporizers consistent with the current subject matter may be referred to by various terms such as inhalable aerosol devices, aerosolizers, vaporization devices, electronic vaping devices, electronic vaporizers, vape pens, etc. Examples of vaporizers consistent with implementations of the current subject matter include electronic vaporizers, electronic cigarettes, e-cigarettes, or the like. In general, such vaporizers are often portable, hand-held devices that heat a vaporizable material to provide an inhalable dose of the material. The vaporizer may include a heater configured to heat a vaporizable material which results in the production of one or more gas-phase components of the vaporizable material. A vaporizable material may include liquid and/or oil-type plant materials, or a semi-solid like a wax, or plant material such as leaves or flowers, either raw or processed. The gas-phase components of the vaporizable material may condense after being vaporized such that an aerosol is formed in a flowing air stream that is deliverable for inhalation by a user.

One or more features of the current subject matter, including one or more of a cartridge (also referred to as a vaporizer cartridge or pod) and a reusable vaporizer device body (also referred to as a vaporizer device base, a body, a vaporizer body, or a base), may be employed with a suitable vaporizable material (where suitable refers in this context to being usable with a device whose properties, settings, etc. are configured or configurable to be compatible for use with the vaporizable material). The vaporizable material may include one or more liquids, such as oils, extracts, aqueous or other solutions, etc., of one or more substances that may be desirably provided in the form of an inhalable aerosol. The cartridge may be inserted into the vaporizer body, and then the vaporizable material heated which results in the inhalable aerosol.

Aspects of the current subject matter relate to heating of the vaporizable material stored in the vaporizer device (e.g., in a vaporizer cartridge). In some implementations, a heater in the vaporizer device includes a heating element that is in thermal contact with the vaporizable material and a nozzle that directs the vaporized vaporizable material to an outlet of the device. Together, the nozzle and the heating element may form a capillary pathway that is capable of causing passive fluid motion (e.g., by capillary action and/or the like) to convey a quantity of the vaporizable material to a region of the vaporizer device (e.g., cartridge) in which the heating element transfers heat to the vaporizable material to vaporize the vaporizable material. That is, the capillary pathway defined by the nozzle and the heating element may draw the vaporizable material from a reservoir (or wicking element) in the vaporizer device such that the vaporizable material may be vaporized by heat delivered from the heating element for subsequent emission from the vaporizer device, for instance, for inhalation by a user in a gas and/or a condensed (e.g., aerosol particles or droplets) phase.

As heating of a vaporizable material is directly correlated with aerosol production, adequate heating of the vaporizable material aids in providing a user a consistent and desired experience. A greater variability in the heating of the vaporizable material when a user puffs on the vaporizer device results in a greater variability in the amount of aerosol produced by the vaporizer device, which may lead to an inconsistent, unsatisfying, and/or undesirable user experience. Greater variability in the heating of the vaporizable material may also make it more difficult to control and/or monitor a precise amount of generated aerosol. Moreover, variability in aerosol production correlates to variability in dosage, which may be of particular concern in medicinal applications. Aspects of the current subject matter provide for improved heating of the vaporizable material for improved aerosol production.

Heating of the vaporizable material may be impeded or otherwise be rendered inefficient by a number of factors including, for example, negative pressure that forms in the cartridge (or device) as the reservoir is depleted of the vaporizable material, re-saturation of vaporizable material within the capillary pathway, high viscosity of the vaporizable material such as various cannabis oils, low surface tension of the vaporizable material, a low capillary force of the capillary pathway, materials of wicks and/or heating elements (e.g., fibrous materials), and/or the like. These factors may, individually or in combination, affect the rate of wicking or re-saturation (e.g., vaporizable material flowing to a region of the device exposed to heat from the heating element, such as the capillary pathway), the consistency of vaporizable material that is vaporized, and control over a precise amount of vaporizable material that is vaporized, as described in detail below.

Aspects of the current subject matter provide for improved heating of vaporizable material by addressing the described limiting factors, including reducing the amount of time to re-saturate the capillary pathway, improving the consistency of produced aerosol, enhancing the ability to control a precise amount of vaporizable material that is vaporized, and improving the ability to monitor the amount of vaporizable material that is vaporized, and remains in the vaporizer device.

Before providing additional details regarding aspects of heating elements in a vaporizer device, the following provides a description of some examples of vaporizer devices including a vaporizer body and a cartridge. The following descriptions are meant to be exemplary, and heating elements consistent with the current subject matter are not limited to the example vaporizer devices described herein.

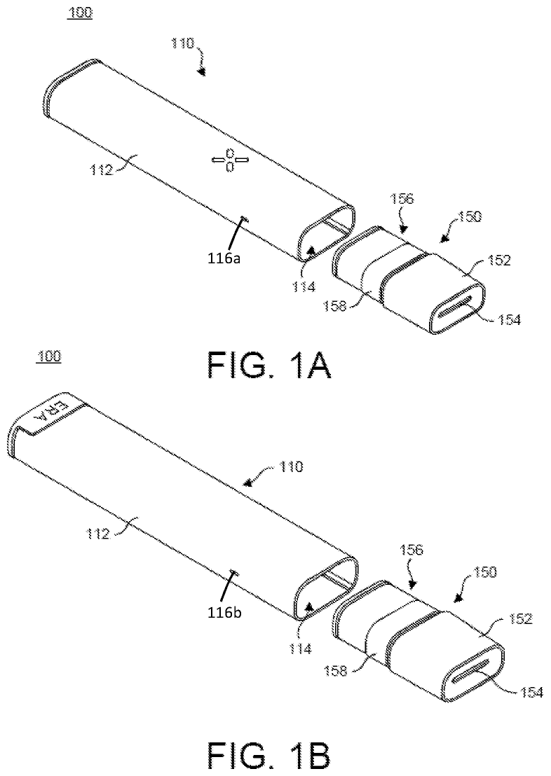

FIG. 1A-FIG. 1F illustrates features of a vaporizer device 100 including a vaporizer body 110 and a cartridge 150 consistent with implementations of the current subject matter. FIG. 1A is a bottom perspective view, and FIG. 1B is a top perspective view of the vaporizer device 100 with the cartridge 150 separated from a cartridge receptacle 114 on the vaporizer body 110. Both of the views in FIG. 1A and FIG. 1B are shown looking towards a mouthpiece 152 of the cartridge 150. FIG. 1C is a bottom perspective view, and FIG. 1D is a top perspective view of the vaporizer device with the cartridge 150 separated from the cartridge receptacle 114 of the vaporizer body 110. FIG. 1C and FIG. 1D are shown looking toward the distal end of the vaporizer body 110. FIG. 1E is top perspective view, and FIG. 1F is a bottom perspective view of the vaporizer device 100 with the cartridge 150 engaged for use with the vaporizer body 110.

As shown in FIG. 1A-FIG. 1D, the cartridge 150 includes, at the proximal end, a mouthpiece 152 that is attached over a cartridge body 156 that forms a reservoir or tank 158 that holds a vaporizable material. The cartridge body 156 may be transparent, translucent, opaque, or a combination thereof. The mouthpiece 152 may include one or more openings 154 (see FIG. 1A, FIG. 1B, FIG. 1F) at the proximal end out of which vapor may be inhaled, by drawing breath through the vaporizer device 100. The distal end of the cartridge body 156 may couple to and be secured to the vaporizer body 110 within the cartridge receptacle 114 of the vaporizer body 110. Power pin receptacles 160a,b (see FIG. 1C, FIG. 1D) of the cartridge 150 mate with respective power pins or contacts 122a,b of the vaporizer body 110 that extend into the cartridge receptacle 114. The cartridge 150 also includes air flow inlets 162a,b on the distal end of the cartridge body 156.

A tag 164, such as a data tag, a near-field communication (NFC) tag, or other type of wireless transceiver or communication tag, may be positioned on at least a portion of the distal end of the cartridge body 156. As shown in FIG. 1C and FIG. 1D, the tag 164 may substantially surround the power pin receptacles 160a,b and the air flow inlets 162a,b, although other configurations of the tag 164 may be implemented as well. For example, the tag 164 may be positioned between the power pin receptacle 160a and the power pin receptacle 160b, or the tag 164 may be shaped as a circle, partial circle, oval, partial oval, or any polygonal shape encircling or partially encircling the power pin receptacles 160a,b and the air flow inlets 162a,b or a portion thereof.

In the example of FIG. 1A, the vaporizer body 110 has an outer shell or cover 112 that may be made of various types of materials, including for example aluminum (e.g., AL6063), stainless steel, glass, ceramic, titanium, plastic (e.g., Acrylonitrile Butadiene Styrene (ABS), Nylon, Polycarbonate (PC), Polyethersulfone (PESU), and the like), fiberglass, carbon fiber, and any hard, durable material. The proximal end of the vaporizer body 110 includes an opening forming the cartridge receptacle 114, and the distal end of the vaporizer body 110 includes a connection 118, such as, for example, a universal serial bus Type C (USB-C) connection and/or the like. The cartridge receptacle 114 portion of the vaporizer body 110 includes one or more openings (air inlets) 116a,b that extend through the outer shell 112 to allow airflow therein, as described in more detail below. The vaporizer body 110 as shown has an elongated, flattened tubular shape that is curvature-continuous, although the vaporizer body 110 is not limited to such a shape. The vaporizer body 110 may take the form of other shapes, such as, for example, a rectangular box, a cylinder, and the like.

The cartridge 150 may fit within the cartridge receptacle 114 by a friction fit, snap fit, and/or other types of secure connection. The cartridge 150 may have a rim, ridge, protrusion, and/or the like for engaging a complimentary portion of the vaporizer body 110. While fitted within the cartridge receptacle 114, the cartridge 150 may be held securely within but still allow for being easily withdrawn to remove the cartridge 150.

Although FIG. 1A-FIG. 1F illustrate a certain configuration of the vaporizer device 100, the vaporizer device 100 may take other configurations as well.

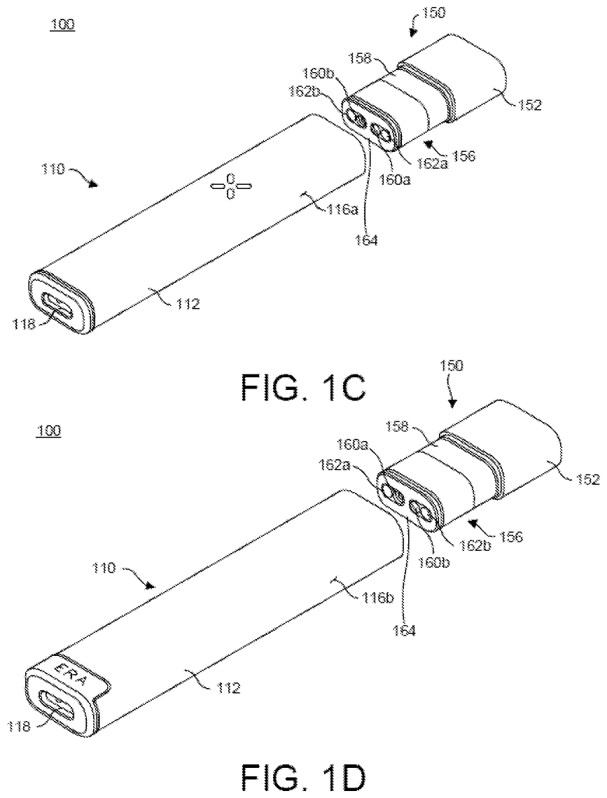

FIG. 2 is a schematic block diagram illustrating components of the vaporizer device 100 having the cartridge 150 and the vaporizer body 110 consistent with implementations of the current subject matter. Included in the vaporizer body 110 is a controller 128 that includes at least one processor and/or at least one memory configured to control and manage various operations among the components of the vaporizer device 100 described herein.

Heater control circuitry 130 of the vaporizer body 110 controls a heater 166 of the cartridge 150. The heater 166 may generate heat to provide vaporization of the vaporizable material. For example, the heater 166 may include a heating coil (e.g., a resistive heater) in thermal contact with a wick which absorbs the vaporizable material, as described in further detail below.

A battery 124 is included in the vaporizer body 110, and the controller 128 may control and/or communicate with a voltage monitor 131 which includes circuitry configured to monitor the battery voltage, a reset circuit 132 configured to reset (e.g., shut down the vaporizer device 100 and/or restart the vaporizer device 100 in a certain state), a battery charger 133, and a battery regulator 134 (which may regulate the battery output, regulate charging/discharging of the battery, and provide alerts to indicate when the battery charge is low, etc.).

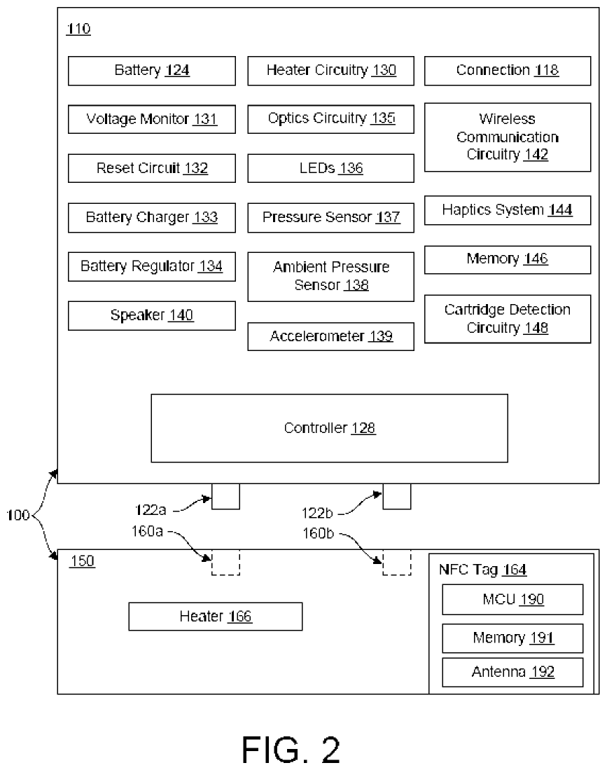

The power pins 122a,b (see also FIG. 4B) of the vaporizer body 110 engage the complementary power pin receptacles 160a,b of the cartridge 150 when the cartridge 150 is engaged with the vaporizer body 110. Alternatively, power pins may be part of the cartridge 150 for engaging complementary power pin receptacles of the vaporizer body 110. The engagement allows for the transfer of energy from an internal power source (e.g., the battery 124) to the heater 166 in the cartridge 150. The controller 128 may regulate the power flow (e.g., an amount or current and/or a voltage amount) to control a temperature at which the heater 166 heats the vaporizable material contained in the reservoir 158. According to implementations of the current subject matter, a variety of electrical connectors other than a pogo-pin and complementary pin receptacle configuration may be used to electrically connect the vaporizer body 110 and the cartridge 150, such as for example, a plug and socket connector.

The controller 128 may control and/or communicate with optics circuitry 135 (which controls and/or communicates with one or more displays such as LEDs 136 which can provide user interface output indications), a pressure sensor 137, an ambient pressure sensor 138, an accelerometer 139, and/or a speaker 140 configured to generate sound or other feedback to a user.

The pressure sensor 137 may be configured to sense a user drawing (e.g., inhaling) on the mouthpiece 152 and activate the heater control circuitry 130 of the vaporizer body 110 to accordingly control the heater 166 of the cartridge 150. In this way, the amount of current supplied to the heater 166 may be varied according the user's draw (e.g., additional current may be supplied during a draw, but reduced when there is not a draw taking place). The ambient pressure sensor 138 may be included for atmospheric reference to reduce sensitivity to ambient pressure changes and may be utilized to reduce false positives potentially detected by the pressure sensor 137 when measuring draws from the mouthpiece 152.

The accelerometer 139 (and/or other motion sensors, capacitive sensors, flow sensors, strain gauge(s), or the like) may be used to detect user handling and interaction, for example, to detect movement of the vaporizer body 110 (such as, for example, tapping, rolling, and/or any other deliberate movement associated with the vaporizer body 110). The detected movements may be interpreted by the controller 128 as one or more predefined user commands. For example, one particular movement may be a user command to gradually increase the temperature of the heater 166 as the user intends to begin using the vaporizer device 100.

The vaporizer body 110, as shown in FIG. 2, includes wireless communication circuitry 142 that is connected to and/or controlled by the controller 128. The wireless communication circuitry 142 may include a near-field communication (NFC) antenna that is configured to read from and/or write to the tag 164 of the cartridge 150. Alternatively or additionally, the wireless communication circuitry 142 may be configured to automatically detect the cartridge 150 as it is being inserted into the vaporizer body 110. In some implementations, data exchanges between the vaporizer body 110 and the cartridge 150 take place over NFC.

The wireless communication circuitry 142 may include additional components including circuitry for other communication technology modes, such as Bluetooth circuitry, Bluetooth Low Energy circuitry, Wi-Fi circuitry, cellular (e.g., LTE, 4G, and/or 5G) circuitry, and associated circuitry (e.g., control circuitry), for communication with other devices. For example, the vaporizer body 110 may be configured to wirelessly communicate with a remote processor (e.g., a smartphone, a tablet, a computer, wearable electronics, a cloud server, and/or processor based devices) through the wireless communication circuitry 142, and the vaporizer body 110 may through this communication receive information including control information (e.g., for setting temperature, resetting a dose counter, etc.) from and/or transmit output information (e.g., dose information, operational information, error information, temperature setting information, charge/battery information, etc.) to one or more of the remote processors.

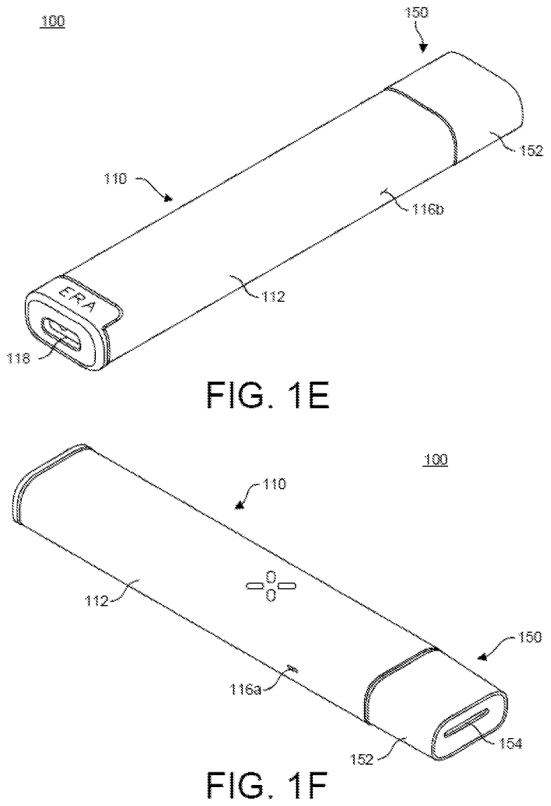

The tag 164 may be a type of wireless transceiver and may include a microcontroller unit (MCU) 190, a memory 191, and an antenna 192 (e.g., an NFC antenna) to perform the various functionalities described below with further reference to FIG. 3. The tag 164 may be, for example, a 1 Kbit or a 2 Kbit tag that is of type ISO/IEC 15693. NFC tags with other specifications may also be used. The tag 164 may be implemented as active NFC, enabling reading and/or writing information via NFC with other NFC compatible devices including a remote processor, another vaporizer device, and/or wireless communication circuitry 142. Alternatively, the tag 164 may be implemented using passive NFC technology, in which case other NFC compatible devices (e.g., a remote processor, another vaporizer device, and/or wireless communication circuitry 142) may only be able to read information from the tag 164.

The vaporizer body 110 may include a haptics system 144, such as an actuator, a linear resonant actuator (LRA), an eccentric rotating mass (ERM) motor, or the like that provide haptic feedback such as a vibration as a “find my device” feature or as a control or other type of user feedback signal. For example, using an app running on a user device (such as, for example, a user device 305 shown in FIG. 3), a user may indicate that he/she cannot locate his/her vaporizer device 100. Through communication via the wireless communication circuitry 142, the controller 128 sends a signal to the haptics system 144, instructing the haptics system 144 to provide haptic feedback (e.g., a vibration). The controller 128 may additionally or alternatively provide a signal to the speaker 140 to emit a sound or series of sounds. The haptics system 144 and/or speaker 140 may also provide control and usage feedback to the user of the vaporizer device 100; for example, providing haptic and/or audio feedback when a particular amount of a vaporizable material has been used or when a period of time since last use has elapsed. Alternatively or additionally, haptic and/or audio feedback may be provided as a user cycles through various settings of the vaporizer device 100. Alternatively or additionally, the haptics system 144 and/or speaker 140 may signal when a certain amount of battery power is left (e.g., a low battery warning and recharge needed warning) and/or when a certain amount of vaporizable material remains (e.g., a low vaporizable material warning and/or time to replace the cartridge 150). Alternatively or additionally, the haptics system 144 and/or speaker 140 may also provide usage feedback and/or control of the configuration of the vaporizer device 100 (e.g., allowing the change of a configuration, such as target heating rate, heating rate, etc.).

The vaporizer body 110 may include circuitry for sensing/detecting when a cartridge 150 is connected and/or removed from the vaporizer body 110. For example, cartridge-detection circuitry 148 may determine when the cartridge 150 is connected to the vaporizer body 110 based on an electrical state of the power pins 122a,b within the cartridge receptacle 114. For example, when the cartridge 150 is present, there may be a certain voltage, current, and/or resistance associated with the power pins 122a,b, when compared to when the cartridge 150 is not present. Alternatively or additionally, the tag 164 may also be used to detect when the cartridge 150 is connected to the vaporizer body 110.

The vaporizer body 110 also includes the connection (e.g., USB-C connection, micro-USB connection, and/or other types of connectors) 118 for coupling the vaporizer body 110 to a charger to enable charging the internal battery 124. Alternatively or additionally, electrical inductive charging (also referred to as wireless charging) may be used, in which case the vaporizer body 110 would include inductive charging circuitry to enable charging. The connection 118 at FIG. 2 may also be used for a data connection between a computing device and the controller 128, which may facilitate development activities such as, for example, programming and debugging.

The vaporizer body 110 may also include a memory 146 that is part of the controller 128 or is in communication with the controller 128. The memory 146 may include volatile and/or non-volatile memory or provide data storage. In some implementations, the memory 146 may include 8 Mbit of flash memory, although the memory is not limited to this and other types of memory may be implemented as well.

The vaporizer device 100 (e.g., cartridge) also includes a vaporizing assembly of vapor-generating components. The vapor-generating components may include the heater 166 configured to heat the vaporizable material to a sufficient temperature that it may vaporize. The vapor-generating components may be arranged as an atomizer or cartomizer or oven. The vapor may be released to a vaporization chamber where the gas phase vapor may condense, forming an aerosol cloud having typical liquid vapor particles with particles having a diameter of average mass of approximately 0.1 micron or greater. In some cases, the diameter of average mass may be approximately 0.1-1 micron.

The heater 166 of the vaporizing assembly may cause the vaporizable material to be converted from a condensed form (e.g., a solid, a liquid, a solution, a suspension, a part of an at least partially unprocessed plant material, etc.) to the gas phase. After conversion of the vaporizable material to the gas phase, and depending on the type of vaporizer, the physical and chemical properties of the vaporizable material, and/or other factors, at least some of the gas-phase vaporizable material may condense to form particulate matter in at least a partial local equilibrium with the gas phase as part of an aerosol, which may form some or all of an inhalable dose provided by the vaporizer device 100 for a given puff or draw on the vaporizer device 100. It will be understood that the interplay between gas and condensed phases in an aerosol generated by a vaporizer may be complex and dynamic, as factors such as ambient temperature, relative humidity, chemistry, flow conditions in airflow paths (both inside the vaporizer and in the airways of a human or other animal), mixing of the gas-phase or aerosol-phase vaporizable material with other air streams, etc., may affect one or more physical parameters of an aerosol.

Vaporizers for use with liquid vaporizable materials (e.g., neat liquids, suspensions, solutions, mixtures, etc.) typically include an atomizer in which a wicking element (also referred to herein as a wick 168), may include any material capable of causing passive fluid motion (for example, by capillary action) to convey an amount of a liquid vaporizable material to a part of the atomizer that includes the heating element. The wicking element is generally configured to draw liquid vaporizable material from the reservoir configured to contain (and that may in use contain) the liquid vaporizable material such that the liquid vaporizable material may be vaporized by heat delivered from the heating element.

The heater 166 may be configured to heat and/or vaporize at least a portion of the vaporizable material drawn towards the heater 166 from the reservoir 158, and may be or include one or more of a conductive heater, a radiative heater, a convective heater, a resistive heater, and/or an inductive heater. One type of vaporizing heating element is a resistive heating element, which may be constructed of or at least include a material (e.g., a metal or alloy, for example a nickel-chromium alloy, or a non-metallic resistor) configured to dissipate electrical power in the form of heat when electrical current is passed through one or more resistive segments of the heating element. A resistive coil or other heating element may be wrapped around, positioned within, integrated into a bulk shape of, pressed into thermal contact with, or otherwise arranged to deliver heat to a wicking element to cause a liquid vaporizable material drawn by the wicking element from a reservoir to be vaporized for subsequent inhalation by a user in a gas and/or a condensed (e.g., aerosol particles or droplets) phase. However, in vaporizer devices having certain types of heaters, it may be difficult to accurately control and/or monitor a quantity of aerosol that is generated and delivered to the user. In certain vaporizer devices, is may also be difficult to provide for consistent aerosol generation. It may also be difficult to supply a small quantity of aerosol or dose of a particular size to the user. In contrast, the heaters consistent with implementations of the current subject matter may provide improved heating of vaporizable material, allowing for accurate and precise delivery of aerosol in small or quantifiable amounts. The heaters described herein may also improve the ability to monitor the amount of aerosol generated and delivered to the user and the amount of vaporizable material remaining in the vaporizer device. The heaters described herein may also provide for more rapid delivery of aerosol to the user, reducing or eliminating the need for a capillary pathway to re-saturate before the delivery of additional aerosol to the user. The heaters described herein may also provide cleaner vaporization of vaporizable material, as the heaters may not include a fibrous wick (e.g., cotton, silica, etc.).

In some implementations, the vaporizer device is compatible with a first type of cartridge having a first heater configuration, such as a wick and heater coil wrapped around the wick, and a second type of cartridge having a second type of heater configuration, such as the heaters 166 described herein, having a nozzle and a heating element. For example, the vaporizer device may be configured such that the vaporizer device has a first mode in which the device is compatible with the first type of cartridge and a second mode in which the device is compatible with the second type of cartridge. In such implementations, the vaporizer device may detect the type of cartridge (e.g., the first type and/or the second type). The vaporizer device may adjust one or more parameters, such as a heating profile, heating temperature, amount of power provided to the heater, and/or the like, to compensate for the type of cartridge coupled with the vaporizer device to help ensure a consistent and improved user experience.

FIG. 3 schematically illustrates a side view of the heater 166 and FIG. 4 schematically illustrates a top view of the heater 166 consistent with implementations of the current subject matter. The heater 166 may be implemented in a vaporizer cartridge, a vaporizer body, or another component of the vaporizer device, in which the heater 166 may be in thermal contact with the vaporizable material. For example, as shown in FIG. 3, the heater 166 may be submerged in, or at least partially submerged in, vaporizable material contained in the reservoir 158. Though FIG. 3 shows an example of the heater 166 fully submerged in the vaporizable material, the heater 166 may be partially submerged in the vaporizable material, or be separately fed the vaporizable material from a separate reservoir or wicking element.

The heater 166 includes a nozzle 202 and a heating element 204. In some implementations, the nozzle 202 and the heating element 204 are integrally formed as a single unit. In other implementations, such as the implementation shown in FIG. 3, the nozzle 202 and the heating element 204 form separate components. In some implementations, the vaporizer cartridge (or device) may also include a vaporizable material supply 206, which may include the reservoir 158 and/or the wicking element 168. For example, the vaporizable material supply 206 may supply vaporizable material to the heater 166 directly from the reservoir 158, without a wick. In some implementations, the vaporizable material supply 206 supplies vaporizable material to the heater 166 via the wicking element 168, which may be formed of any of a variety of materials, including metals, polymer, natural fibers, synthetic fibers, silica fibers, cotton, ceramic, hemp, stainless steel mesh, rope cables, and/or any porous medium, such as for example sintered glass beads, or another absorbent material that is configured to hold vaporizable material to be vaporized by the heating element 204. In other implementations, the vaporizable material supply 206 supplies vaporizable material to the heater 166 both directly from the reservoir 158 and from the wicking element 168.

The heating element 204 of the heater 166 includes two conductive elements 210 (e.g., plates, materials, and/or the like). In other implementations, the conductive elements 210 include three, four, five, six, or more conductive elements. The conductive elements 210 may include copper, stainless steel, ceramic, and/or another conductive material.

The conductive elements 210 are spaced apart from one another to form an opening 212 therebetween. The opening 212 may have a diameter that allows the conductive elements 210 to be spaced apart from one another, yet be positioned close enough to conduct electricity between the conductive elements 210 across the opening 212. In other words, the pair of conductive elements 210 may be close enough to arc electricity across the conductive elements 210 at a given potential. Additionally and/or alternatively, the pair of conductive elements 210 may form a capacitor such that the electric arc is a capacitive discharge between the two conductive elements 210. The diameter of the opening 212 may also be sufficiently sized to draw and/or hold at least some vaporizable material into the opening 212 from the wicking element 206 (or reservoir 158), for example, via capillary action. The diameter of the opening 212 may be approximately 0.15 mm, 2.0 mm, 0.15 mm to 2.0 mm, 0.15 mm to 0.50 mm, 0.50 mm to 1.0 mm, 1.0 mm to 1.5 mm, 1.5 mm to 2.0 mm or larger.

In some implementations, the conductive elements 210 include a first conductive element 210A and a second conductive element 210B. The first conductive element 210A may be positively charged and the second conductive element 210B may be negatively charged. Alternatively, the first conductive element 210A may be negatively charged and the second conductive element 210B may be positively charged. As noted above, when power is supplied to the heating element 204, electricity may arc between the first conductive element 210A and the second conductive element 210B, thereby causing vaporization of the vaporizable material located between the first and second conductive elements 210A, 210B (e.g., within, around, and/or adjacent to the opening 212).

The nozzle 202 may direct the vaporized vaporizable material to an outlet of the vaporizer cartridge (or device). For example, the nozzle 202 may have a channel 214 that extends from a distal end to a proximal end of the nozzle 202. The channel 214 may define a hollow tube (e.g., an interior volume) that extends along a central longitudinal axis of the nozzle 202. The channel 214 may be sized to draw and/or hold vaporizable material from the reservoir 158, wicking element 206, and/or opening 212, such as via capillary action. As shown in FIG. 3, the vaporizable material may have a meniscus 211 towards the proximal end of the channel 214 due to the capillary action caused by the shape of the channel 214. In some implementations, the diameter of the channel 214 is the same as the diameter of the opening 212. The diameter of the channel may have a diameter of approximately 0.15 mm, 2.0 mm, 0.15 mm to 2.0 mm, 0.15 mm to 0.50 mm, 0.50 mm to 1.0 mm, 1.0 mm to 1.5 mm, 1.5 mm to 2.0 mm or larger. The diameter of the channel 214 (and/or the opening 212) may, in some implementations of the current subject matter, be particularly adapted for use with an oil-based vaporizable material, such as cannabis-derived oils, although other types of vaporizable materials may be used as well. For example, in some implementations, the diameter of the channel 214 is sized depending on the type of vaporizable material stored in the device—the diameter of the channel 214 may be wider when used with a vaporizable material having a relatively high surface tension because lesser capillary force would be required to draw and/or hold the vaporizable material within the channel 214. Alternatively, the diameter of the channel 214 may be narrower when used with a vaporizable material having a relatively low surface tension because greater capillary force would be required to draw and/or hold the vaporizable material within the channel 214.

The nozzle 202 may be positioned adjacent to the heating element 204 on one or more sides of the heating element 204. In some implementations, the distal side of the nozzle 202 abuts the proximal side of the heating element 204. In some implementations, the nozzle 202 is spaced apart from the heating element, as described in more detail below.

The nozzle 202 and the heating element 204 may be axially aligned along a longitudinal axis 230 of the heater 166. In the example described above, the channel 214 of the nozzle 202 is centrally aligned along the axis 230 with the opening 212 formed between the conductive elements 210 of the heating element 204. This alignment allows for the heating element 204 to vaporize an amount of the vaporizable material, and for the vaporized vaporizable material to be directed through the channel 214 to the outlet of the vaporizer device to be inhaled by the user. The channel 214 and the opening 212 may together or separately form a capillary pathway that is generally large enough to permit wicking of sufficient vaporizable material to replace vaporized liquid transferred from the reservoir 158 by capillary action during vaporization, but may be small enough to prevent leakage of the vaporizable material out of the capillary pathway during normal operation.

The heater 166 advantageously helps to provide improved and more precise dosing by vaporizing controlled amounts of the vaporizable material. The heater 166 may do so without the use of a resistive heater wire wrapped around a fibrous wick.

The held vaporizable material within the capillary pathway may be heated and vaporized for inhalation by the user. In operation, after the vaporizer device 100 is fully charged (or in some instances partially charged), a user may activate the vaporizer device 100 by drawing (e.g., inhaling) through the mouthpiece. The vaporizer device 100 may detect a draw (e.g., using one or more pressure sensors, flow sensors, microphones, and/or the like, including a sensor configured to detect a change in temperature or power applied to a heater element) and may increase the power to a predetermined temperature preset to supply power to the heating element 204. When power is supplied to the heating element 204, such as to the first and/or second conductive elements 210A, 210B, electricity is arced at a voltage (e.g., a high voltage) between the first and second conductive elements 210A, 210B, across the vaporizable material (within the opening 212 and/or within the channel 214). The electric arc (e.g., an electric and/or a capacitive discharge) transfers energy into the vaporizable material, thereby causing the vaporizable material in contact with the electric arc to vaporize and generating an inhalable aerosol. As the vaporizable material is vaporized and is directed through the channel 214 to the outlet of the vaporizer device, a vacuum may be created in the opening 212 and/or the channel 214. The vacuum and/or the capillary force provided by the nozzle 202 draws vaporizable material from the vaporizable material supply 206 to refill the opening 212 and/or the channel 214. Depending on certain factors, such as dose size, session size, the number of puffs taken by the user, and the like, electricity may continuously, intermittently, and/or repeatedly be arced across the conductive elements 210 of the heating element 204 to repeatedly and rapidly produce the inhalable aerosol to be inhaled by the user.

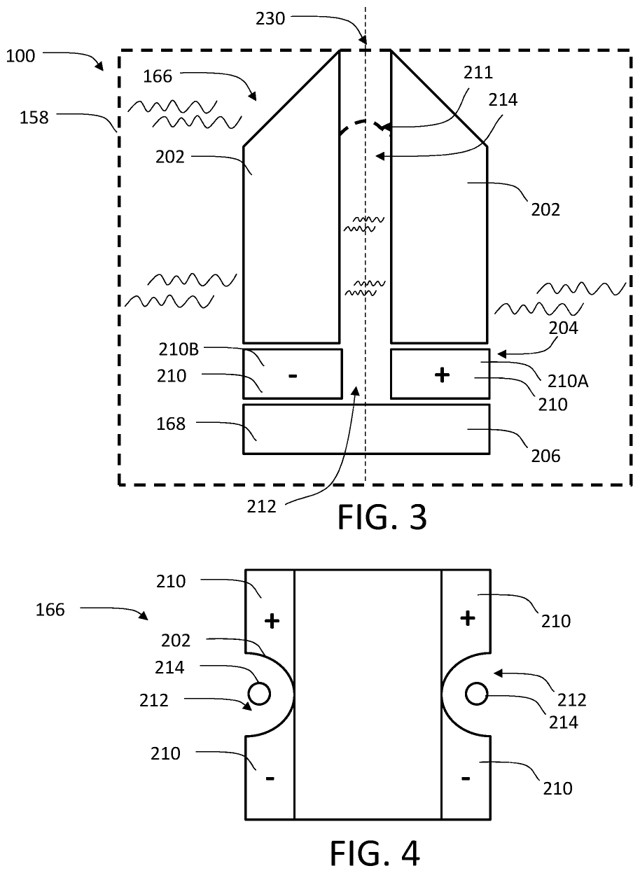

In some implementations of the current subject matter, the vaporizer device may include a plurality of heaters 166 (e.g., a plurality of nozzles 202 and a plurality of heating elements 204 that correspond with each of the plurality of nozzles 202). FIG. 5 illustrates an example of the vaporizer device having a plurality of heaters 166 consistent with implementations of the current subject matter. As shown in FIG. 5, the plurality of heaters 166 includes a plurality of nozzles 202 positioned on or forming a part of a nozzle substrate 220. In some implementations, the plurality of nozzles 202 are arranged in an array or other patterned arrangement on the nozzle substrate 220.

Similarly, the plurality of heaters 166 shown in FIG. 5 includes a plurality of heating elements 204 (each of which includes the conductive elements 210) that correspond with each of the plurality of nozzles 202. The plurality of heating elements 204 are positioned on and/or form a part of an energy transfer substrate 222. In some implementations, the opening 212 forms a channel extending through a thickness of the energy transfer substrate 222, between the conductive elements 210 of each of the plurality of heating elements 204. The conductive elements 210 of each of the plurality of heating elements may surround each of the openings 210 in the energy transfer substrate 222.

The nozzle substrate 220 may abut or otherwise be pressed against the energy transfer substrate 222. When assembled, the nozzle substrate 220 is aligned with the energy transfer substrate 222 such that each of the plurality of nozzles 202 (e.g., the channel 214 of each of the plurality of nozzles 202) is axially aligned with each of the plurality of heating elements 204 (e.g., the opening 212 formed between the conductive elements 210 of each of the plurality of heating elements 204). In this arrangement in which the vaporizer device includes a plurality of heaters 166, power may be supplied to one, two, or more of the heaters 166 (e.g., the heating elements 204) simultaneously to heat the one, two, or more heaters 166 at the same time. In other implementations, power may be supplied to one, two, or more of the heaters 166 in a patterned sequence (e.g., sequentially, staggered sequence, by row of the array of heaters, by column of the array of heaters, etc.). For example, power may be supplied to a first heating element 204A of a first heater 166A or a first subset of heating elements 204A, causing vaporization of at least a portion of the vaporizable material held within a first opening 212A and/or a first channel 214A of a first corresponding nozzle 202A. After or during vaporization of the vaporizable material held within the first heater 166A, power may be supplied to a second heating element 204B of a second heater 166B or a second subset of heating elements 204B, causing vaporization of at least a portion of the vaporizable material held within a second opening 212B and/or a second channel 214B of a second corresponding nozzle 202B, and so on.

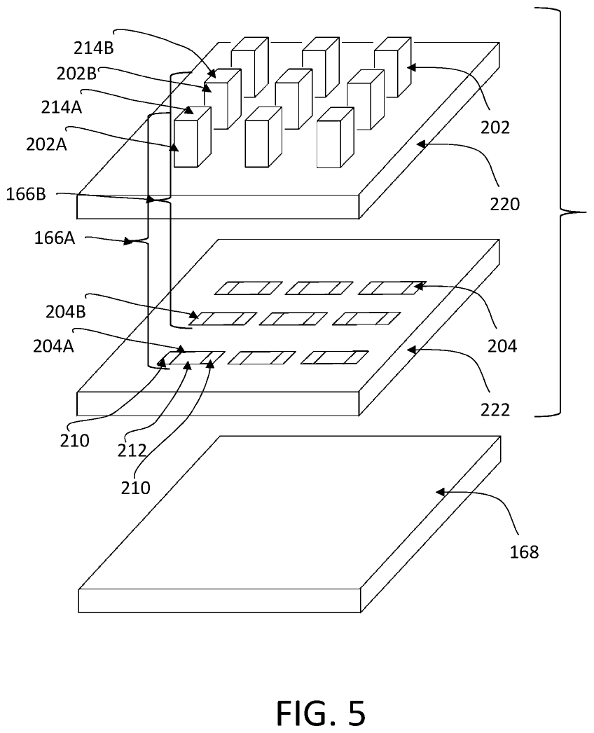

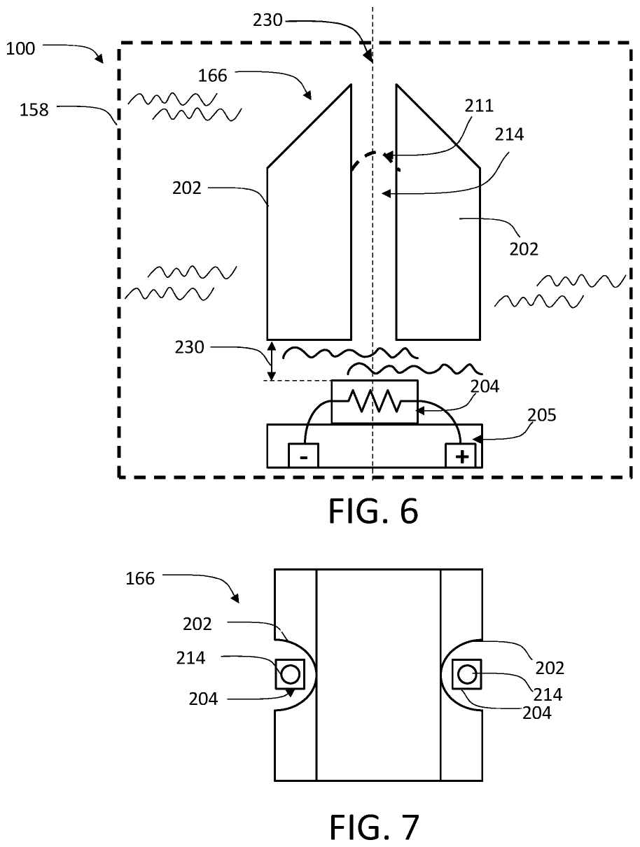

FIGS. 6-8 illustrate another example of the heater 166 consistent with implementations of the current subject matter. FIG. 6 schematically illustrates a side view of the heater 166 and FIG. 7 schematically illustrates a top view of the heater 166 consistent with implementations of the current subject matter. Similar to the example heater 166 shown in FIG. 166, the heater 166 includes a nozzle 202 (including the channel 214) and a heating element 204, which may be implemented in a vaporizer cartridge, a vaporizer body, or another component of the vaporizer device, in which the heater 166 may be in thermal contact with the vaporizable material. For example, as shown in FIG. 6, the heater 166 may be submerged in, or at least partially submerged in vaporizable material contained in the reservoir 158. In some implementations, the nozzle 202 and the heating element 204 are integrally formed as a single unit. In other implementations, such as the implementation shown in FIG. 6, the nozzle 202 and the heating element 204 form separate components.

As shown in FIG. 6, the heating element 204 may include a conductive element, resistive heating element, or a MEMS heating element, coupled to a substrate, such as a printed circuit board (PCB) 205. The PCB 205 can electrically and structurally support the heating element 204. In some implementations, the PCB 205 includes a controller that determines when to supply power to the heating element 204, such as, for example, when the user is going to or is taking a puff (e.g., when the pressure sensor detects a change in pressure). The controller may cause an amount of power to be supplied to the heating element 204 based on the change in pressure. The controller may also determine the number of or sequence in which the heaters are supplied power based upon user input, a required amount of aerosol, etc., as explained in more detail below.

As shown in FIG. 6, the heating element 204 is centrally aligned with the longitudinal axis 230 of the channel 214 of the nozzle 202 to allow the vaporized vaporizable material to be directed through the channel 214 to the outlet of the vaporizer device 100. In some implementations, the heating element 204 is spaced apart from the nozzle 202 by a gap 230. The gap 230 may have a length that is the same as or similar to the diameter of the channel 214, to improve the re-saturation of the channel 214 with vaporizable material from the reservoir 158 via a capillary force. This alignment allows for the heating element 204 to vaporize an amount of the vaporizable material, and for the vaporized vaporizable material to be directed through the channel 214 to the outlet of the vaporizer device to be inhaled by the user. For example, in some implementations, the diameter of the channel 214 is the same as the length of the gap 230. The diameter of the channel 214 may have a diameter of approximately 0.15 mm, 2.0 mm, 0.15 mm to 2.0 mm, 0.15 mm to 0.50 mm, 0.50 mm to 1.0 mm, 1.0 mm to 1.5 mm, 1.5 mm to 2.0 mm or larger or smaller. The diameter of the channel 214 (and/or the length of the gap 230) may, in some implementations of the current subject matter, be particularly adapted for use with an oil-based vaporizable material, such as cannabis-derived oils, although other types of vaporizable materials may be used as well.

The heater 166 advantageously helps to provide improved and more precise dosing by vaporizing controlled amounts of the vaporizable material. The heater 166 may do so without the use of a resistive heater wire wrapped around a wick. As noted above, the channel 214 may form a capillary passageway that draws and holds an amount of vaporizable material from the vaporizable material supply 206 (e.g., the reservoir 158). The held vaporizable material may be heated and vaporized for inhalation by the user. For example, consistent with implementations of the current subject matter, in operation, after the vaporizer device 100 is fully charged (or in some instances partially charged), a user may activate the vaporizer device 100 by drawing (e.g., inhaling) through the mouthpiece. The vaporizer device 100 may detect a draw (e.g., using one or more pressure sensors, flow sensors, microphones, and/or the like, including a sensor configured to detect a change in temperature or power applied to a heater element) and may increase the power to a predetermined temperature preset to supply power to the heating element 204.

When power is supplied to the heating element 204, the heating element 204 transfers heat to the vaporizable material within the capillary pathway (e.g., in contact with the heating element 204 and/or that is positioned within the gap 230 and/or the channel 214), thereby vaporizing the vaporizable material. The vaporized vaporizable material is directed through the channel 214 to the outlet of the vaporizer device. As the vaporized vaporizable material exits the channel 214, a vacuum is created in the channel 214. The vacuum and/or the capillary force provided by the nozzle 202 draws vaporizable material from the vaporizable material supply 206 to refill the channel 214. Depending on certain factors, such as dose size, session size, the number of puffs taken by the user, and the like, electricity may continuously and/or repeatedly be supplied to the heating element 204 to repeatedly and rapidly produce an inhalable aerosol to be inhaled by the user.

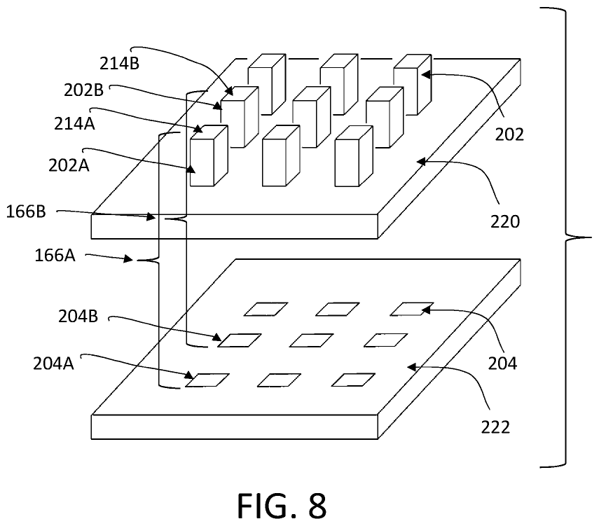

In some implementations of the current subject matter, the vaporizer device 100 may include a plurality of heaters 166 (e.g., a plurality of nozzles 202 and a plurality of heating elements 204 that correspond with each of the plurality of nozzles 202). FIG. 8 illustrates an example of the vaporizer device having a plurality of heaters 166 consistent with implementations of the current subject matter. As shown in FIG. 8, the plurality of heaters 166 includes a plurality of nozzles 202 that are positioned on or form a part of a nozzle substrate 220. In some implementations, the plurality of nozzles 202 are arranged in an array or other patterned arrangement on the nozzle substrate 220. The plurality of heaters 166 shown in FIG. 8 also each include a heating element 204 (and PCB 205) that corresponds with each nozzle of the plurality of nozzles 202. Each of the heating elements 204 and PCBs 205 are positioned on and/or forms a part of an energy transfer substrate 222. For example, in some implementations, the energy transfer substrate 222 forms the PCB 205, and the heating elements 204 are coupled to the energy transfer substrate 222.

The nozzle substrate 220 may abut or otherwise be pressed against the energy transfer substrate 222. When assembled, the nozzle substrate 220 is aligned with the energy transfer substrate 222 such that each of the plurality of nozzles 202 (e.g., the channel 214 of each of the plurality of nozzles 202) is axially aligned with each of the plurality of heating elements 204. In this arrangement in which the vaporizer device 100 includes a plurality of heaters 166, power may be supplied to one, two, or more of the heaters 166 (e.g., the heating elements 204) simultaneously to heat the one, two, or more heaters 166 at the same time. In other implementations, power may be supplied to one, two, or more of the heaters 166 in a patterned sequence (e.g., sequentially, staggered sequence, row by row of the array of heaters, and/or column by column of the array of heaters). For example, power may be supplied to a first heating element 204A of a first heater 166A or a first subset of heating elements 204A, causing vaporization of the vaporizable material held within a first channel 214A of a first corresponding nozzle 202A. After or during vaporization of the vaporizable material held within the first heater 166A, power may be supplied to a second heating element 204B of a second heater 166B or a second subset of heating elements 204B, causing vaporization of the vaporizable material held within a second channel 214B of a second corresponding nozzle 202B, and so on.

Supplying power to each of the plurality of heating elements 204 in the array of heating elements in a sequence (e.g., sequentially) advantageously allows for faster generation of aerosol (e.g., vaporized vaporizable material), as the amount of time waiting for a wick to re-saturate is reduced or eliminated. For example, vaporizable material held within a second heater (or subset of heaters) can be vaporized while the first heater (or subset of heaters) is vaporizing vaporizable material or is re-saturated with the vaporizable material. Supplying power to each of the heaters described herein individually, or in a particular sequence, also advantageously improves the accuracy, control, consistency, and monitoring of the amount of vaporizable material that is vaporized and provided to the user for inhalation. For example, each capillary tube (formed by the opening 212 and/or the channel 214) holds a known amount of vaporizable material. Thus, when the vaporizable material in each capillary tube is vaporized, the quantity of vaporized vaporizable material and generated aerosol is also known. These implementations can help improve the monitoring of the amount of vaporizable material that is vaporized, the quantity of vaporizable material remaining in the vaporizable material supply, and other characteristics of the generated aerosol. These implementations may also improve the ability to generate smaller known quantities of aerosol which may form some or all of an inhalable dose provided by the vaporizer device 100 for a given puff or draw on the vaporizer device 100.

Additionally, in some implementations, the number and sequence of heaters being supplied power to vaporize the vaporizable material held within each corresponding heater can be controlled. Thus, the vaporizable material held within all or a subset of heaters can be vaporized depending on a number of factors as described herein. As noted above, in some implementations, the heater (or vaporizer device) includes a controller that determines the number of or sequence in which heaters are supplied power based upon user input, a required amount of aerosol, etc.

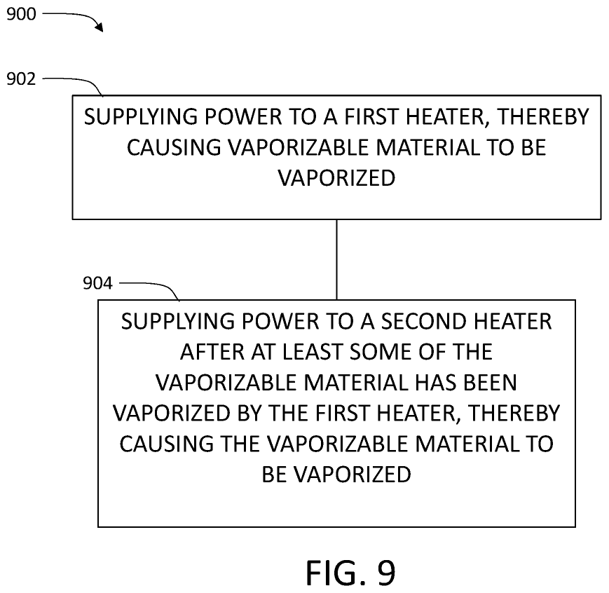

FIG. 9 illustrates an example method 900 of heating a vaporizable material stored within a vaporizer device, using one or more of the heaters described above, consistent with implementations of the current subject matter. At 902, power may be supplied to the first heater 166A of the vaporizer device 100, thereby causing the vaporizable material in thermal contact with the first heater to be vaporized, as described above. At 904, power may be supplied to the second heater of the vaporizer device after at least some of the vaporizable material in thermal contact with the first heater has been vaporized, thereby causing the vaporizable material in thermal contact with the second heater 166B to be vaporized. In some implementations, power may be supplied to any number of heaters in various sequences to generate an aerosol.

In some examples, the vaporizable material may include a viscous liquid such as, for example a cannabis oil. In some variations, the cannabis oil comprises between 0.3% and 100% cannabis oil extract. The viscous oil may include a carrier for improving vapor formation, such as, for example, propylene glycol, glycerol, medium chain triglycerides (MCT) including lauric acid, capric acid, caprylic acid, caproic acid, etc., at between 0.01% and 25% (e.g., between 0. 1% and 22%, between 1% and 20%, between 1% and 15%, and/or the like). In some variations the vapor-forming carrier is 1,3-Propanediol. A cannabis oil may include a cannabinoid or cannabinoids (natural and/or synthetic), and/or a terpene or terpenes derived from organic materials such as for example fruits and flowers. For example, any of the vaporizable materials described herein may include one or more (e.g., a mixture of) cannabinoid including one or more of: CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), Tetrahydrocannabinol, Cannabidiol (CBD), Cannabinol (CBN), Tetrahydrocannabinolic Acid (THCA), Cannabidioloc Acid (CBDA), Tetrahydrocannabivarinic Acid (THCVA), one or more Endocannabinoids (e.g., anandamide, 2-Arachidonoylglycerol, 2-Arachidonyl glyceryl ether, N-Arachidonoyl dopamine, Virodhamine, Lysophosphatidylinositol), and/or a synthetic cannabinoids such as, for example, one or more of: JWH-018, JWH-073, CP-55940, Dimethylheptylpyran, HU-210, HU-331, SR144528, WIN 55,212-2, JWH-133, Levonantradol (Nantrodolum), and AM-2201. The oil vaporization material may include one or more terpene, such as, for example, Hemiterpenes, Monoterpenes (e.g., geraniol, terpineol, limonene, myrcene, linalool, pinene, Iridoids), Sesquiterpenes (e.g., humulene, farnesenes, farnesol), Diterpenes (e.g., cafestol, kahweol, cembrene and taxadiene), Sesterterpenes, (e.g., geranylfarnesol), Triterpenes (e.g., squalene), Sesquarterpenes (e.g, ferrugicadiol and tetraprenylcurcumene), Tetraterpenes (lycopene, gamma-carotene, alpha- and beta-carotenes), Polyterpenes, and Norisoprenoids. For example, an oil vaporization material as described herein may include between 0.3-100% cannabinoids (e.g., 0.5-98%, 10-95%, 20-92%, 30-90%, 40-80%, 50-75%, 60-80%, etc.), 0-40% terpenes (e.g., 1-30%, 10-30%, 10-20%, etc.), and 0-25% carrier (e.g., medium chain triglycerides (MCT)).

In any of the oil vaporizable materials described herein (including in particular, the cannabinoid-based vaporizable materials), the viscosity may be within a predetermined range. The range may be between, at room temperature (23° C.) about 30 cP (centipoise) and 115 kcP (kilocentipoise), between 30 cP and 200 kcP, although higher viscosities and/or lower viscosities may be implemented as well. For example, the viscosity may be between 40 cP and 113 kcP at room temperature. Outside of this range, the vaporizable material may fail in some instances to wick appropriately to form a vapor as described herein. In particular, it is typically desired that the oil may be made sufficiently thin to both permit wicking at a rate that is useful with the apparatuses described herein, while also limiting leaking (e.g., viscosities below that of ˜30 cP at room temperature might result in problems with leaking).

Although the disclosure, including the figures, described herein may described and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A vaporizer device, comprising:
a reservoir configured to store a vaporizable material; and
a heater configured to vaporize the vaporizable material stored in the reservoir, the heater comprising:
a nozzle comprising a channel extending along a central longitudinal axis, the channel configured to direct the vaporized vaporizable material to an outlet of the vaporizer device;
a heating element comprising a pair of conductive elements that are separated by an opening, the opening being axially aligned with the channel along the central longitudinal axis, the pair of conductive elements being oppositely charged; and
a capillary pathway defined by the channel and the opening, the capillary pathway configured to draw and hold the vaporizable material;
wherein an electric arc is configured to form between the pair of conductive elements when power is supplied to the heating element, thereby vaporizing at least some of the vaporizable material held within the capillary pathway.

2. The vaporizer device of claim 1, further comprising a plurality of heaters positioned in a patterned array.

3. The vaporizer device of any of the preceding claims, wherein the heater further comprises a nozzle substrate and a heat transfer substrate.

4. The vaporizer device of claim 3, wherein a plurality of nozzles are positioned on the nozzle substrate and a plurality of heating elements are positioned on the heat transfer substrate.

5. The vaporizer device of claim 2, wherein the plurality of heaters comprises a first heater and a second heater.

6. The vaporizer device of claim 5, wherein power is supplied sequentially to the first heater and the second heater such that the second heater vaporizes the vaporizable material held within the capillary pathway of the second heater after the first heater vaporizes at least some of the vaporizable material held within the capillary pathway of the first heater.

7. The vaporizer device of claim 3, wherein the opening defines a passageway formed through a thickness of the heat transfer substrate.

8. The vaporizer device of any of claims 2 to 7, wherein the nozzle abuts the heating element.

9. A heater for a vaporizer device configured to vaporize vaporizable material stored within a reservoir of the vaporizer device, the heater comprising:
a nozzle comprising a channel extending along a central longitudinal axis, the channel configured to direct vaporized vaporizable material to an outlet of the vaporizer device;
a heating element comprising a pair of conductive elements that are separated by an opening, the opening being axially aligned with the channel along the central longitudinal axis, the pair of conductive elements being oppositely charged; and
a capillary pathway defined by the channel and the opening, the capillary pathway configured to draw and hold the vaporizable material;
wherein an electric arc is configured to form between the pair of conductive elements when power is supplied to the heating element, thereby vaporizing at least some of the vaporizable material held within the capillary pathway.

10. A vaporizer device, comprising:
a reservoir configured to store a vaporizable material; and
a heater configured to vaporize the vaporizable material stored in the reservoir, the heater comprising:
a nozzle comprising a channel extending along a central longitudinal axis, the channel configured to direct vaporized vaporizable material to an outlet of the vaporizer device;
a heating element spaced apart from the nozzle by a gap, the heating element comprising a resistive element configured to generate heat, the heating element being axially aligned with the channel along the central longitudinal axis;
a printed circuit board coupled to and supporting the heating element, the printed circuit board comprising a controller configured to control the generation of heat by the heating element; and
a capillary pathway defined by the channel and the gap, the capillary pathway configured to draw and hold the vaporizable material;
wherein the heat generated by the heating element is transferred to the vaporizable material held within the capillary pathway, thereby causing the vaporization of at least some of the vaporizable material.

11. The vaporizer device of claim 10, further comprising a plurality of heaters positioned in a patterned array.

12. The vaporizer device of any of claims 10 to 11, wherein the heater further comprises a nozzle substrate and a heat transfer substrate.

13. The vaporizer device of claim 12, wherein a plurality of nozzles are positioned on the nozzle substrate and a plurality of heating elements are positioned on the heat transfer substrate.

14. The vaporizer device of claim 11, wherein the plurality of heaters comprises a first heater and a second heater.

15. The vaporizer device of claim 14, wherein power is supplied sequentially to the first heater and the second heater such that the second heater vaporizes the vaporizable material held within the capillary pathway of the second heater after the first heater vaporizes at least some of the vaporizable material held within the capillary pathway of the first heater.

16. A heater for a vaporizer device configured to vaporize vaporizable material stored within a reservoir of the vaporizer device, the heater comprising:
a nozzle comprising a channel extending along a central longitudinal axis, the channel configured to direct vaporized vaporizable material to an outlet of the vaporizer device;
a heating element spaced apart from the nozzle by a gap, the heating element comprising a resistive element configured to generate heat, the heating element being axially aligned with the channel along the central longitudinal axis;
a printed circuit board coupled to and supporting the heating element, the printed circuit board comprising a controller configured to control the generation of heat by the heating element; and
a capillary pathway defined by the channel and the gap, the capillary pathway configured to draw and hold the vaporizable material;
wherein the heat generated by the heating element is transferred to the vaporizable material held within the capillary pathway, thereby causing the vaporization of at least some of the vaporizable material.

17. A method of vaporizing a vaporizable material stored within a vaporizer device, the method comprising:
supplying power to a first heater of the vaporizer device, thereby causing the vaporizable material in thermal contact with the first heater to be vaporized; and
supplying power to a second heater of the vaporizer device after at least some of the vaporizable material in thermal contact with the first heater has been vaporized, thereby causing the vaporizable material in thermal contact with the second heater to be vaporized.

18. The method of claim 17, wherein each of the first heater and the second heater comprises:
a nozzle comprising a channel extending along a central longitudinal axis, the channel configured to direct vaporized vaporizable material to an outlet of the vaporizer device;
a heating element comprising a pair of conductive elements that are separated by an opening, the opening being axially aligned with the channel along the central longitudinal axis, the pair of conductive elements being oppositely charged; and
a capillary pathway defined by the channel and the opening, the capillary pathway configured to draw and hold the vaporizable material;
wherein an electric arc is configured to form between the pair of conductive elements when power is supplied to the heating element, thereby vaporizing at least some of the vaporizable material held within the capillary pathway.

19. The method of claim 17, wherein each of the first heater and the second heater comprises:
a nozzle comprising a channel extending along a central longitudinal axis, the channel configured to direct the vaporized vaporizable material to an outlet of the vaporizer device;
a heating element spaced apart from the nozzle by a gap, the heating element comprising a resistive element configured to generate heat, the heating element being axially aligned with the channel along the central longitudinal axis;
a printed circuit board coupled to and supporting the heating element, the printed circuit board comprising a controller configured to control the generation of heat by the heating element; and
a capillary pathway defined by the channel and the gap, the capillary pathway configured to draw and hold the vaporizable material;
wherein the heat generated by the heating element is transferred to the vaporizable material held within the capillary pathway, thereby causing the vaporization of at least some of the vaporizable material.
